(19) United States
(12) Patent Application Publication
(10) Pub. No.: US 2005/0079608 A1
(43) Pub. Date: Apr. 14, 2005

(54) Lineage committed stem cells selected for telomerase promoter activity

(76) Inventors: Karl L. Skorecki, Kiryat Shmuel (IL); Maty Tzukerman, M.P. Western Galilee (IL)

Correspondence Address: WINSTON & STRAWN;PATENT DEPARTMENT, 1400 L STREET, N.W., WASHINGTON, DC, 20005-3502, US

(21) Appl. No.: 10/911,682

(22) Filed: Aug. 4, 2004

Related U.S. Application Data
(63) Continuation of application No. PCT/IL03/00091, filed on Feb. 5, 2003.
(60) Provisional application No. 60/353,996, filed on Feb. 5, 2002.

(51) Int. Cl. C12N 5/08; C12Q 1/68

(52) U.S. Cl. 435/366; 435/6

(57) ABSTRACT

The present invention discloses transfected embryonic stem cells capable of providing telomerase positive progenitor cells that are capable of proliferation and maintain telomerase gene promoter activity, and to the isolation and propagation of such populations useful for cell replacement therapy. Improvements in cell replacement therapy utilizing such cells form another embodiment of the invention.

### CROSS-REFERENCE TO RELATED APPLICATIONS

This application is a continuation of International application PCT/IL03/00091 filed Feb. 5, 2003, and claims the benefit of US provisional application 60/353,996 filed Feb. 5, 2002, the entire content of each if which is expressly incorporated herein by reference thereto.

### FIELD OF THE INVENTION

The present invention relates to transfected embryonic stem cells capable of differentiating into progenitor cells that express telomerase, proliferate and maintain telomerase gene promoter activity, and to the isolation and propagation of such progenitor cells for use in cell replacement therapy.

### BACKGROUND OF THE INVENTION

Pluripotent human embryonic stem cells (HESC) are capable of differentiating into many cell types, which makes them and their derivatives proper candidates for research and medical applications, including cellular transplantation. The transition of cells from a pluripotent to a differentiated state during the process of organogenesis is accompanied with shortening of chromosome ends, also called telomeres.

Preservation of the integrity of chromosome ends, and hence genome stability, requires the activity of the ribonucleoprotein enzyme telomerase. This enzyme is active in the earliest stages of embryonic development, whereas during subsequent stages of fetal development its activity is suppressed. Human embryonic stem cells in culture also exhibit a reduction in telomerase activity with the transition from growth conditions to conditions enabling their differentiation. This loss of telomerase activity with differentiation and the resulting limited life span of the terminally differentiated cells severely reduces the utility of hESC, for example as a source for cell replacement therapy. The modes of growth arrest associated with terminal differentiation are (a) Go arrest that is not related to senescence; and (b) replicative arrest related to senescence, which limits the total number of rounds of replication.

Ectopic or exogenous telomerase over-expression may be used to overcome senescence.

Stem Cells Populations and Methods of Culturing Thereof

Methods for generating embryonic cell populations and methods for propagation and for immortalization of this type of cells are known in the art. For example, pluripotent hESC that maintain their multipotential capacity to differentiate into various cell types and methods for isolation, culture and expansion of such cells utilizing combinations of growth factors, are disclosed in U.S. Pat. Nos. 5,690,926; 5,753,506 and in the European Patent Application No. 380646 among many others.

A method of enriching a population of mammalian cells for stem cells is disclosed in U.S. Pat. No. 6,146,888. The method comprises the steps of: providing in vitro a mixed population of mammalian cells whose genome comprises at least one nucleic acid construct encoding an antibiotic resistance gene operatively linked to a promoter which preferentially expresses said antibiotic gene in mammalian stem cells, culturing said mixed population of mammalian cells in vitro under conditions conducive to cell survival wherein the preferential expression of said antibiotic resistance gene results in the preferential survival of mammalian stem cells in the presence of antibiotic.

A method for culturing human embryonic stem cells in vitro for prolonged maintenance while preserving the pluripotent character of these cells, as well as a purified preparation of said cells, is disclosed in U.S. Pat. No. 6,200,806. It is further disclosed that these embryonic stem cells also retain the ability, throughout the culture and after continuous culture for eleven months, to differentiate into all tissues derived from all three embryonic germ layer.

A method for selective ex-vivo expansion of stein cells is disclosed in U.S. Pat. No. 6,479,261. The method comprises the steps of separating stem cells from other cells and culturing the separated stem cells ir a growth media comprising a modified human interleukin-3 polypeptide having at least three times greater cell proliferative activity than native human interleukin-3, in at least one assay selected from the group consisting of: AML cell proliferation, TF-1 cell proliferation and Methylcellulose assay.

A population of HESC which under appropriate culture conditions differentiate into a substantially high percentage of insulin producing cells in spontaneously formed aggregated embryoid bodies is disclosed in International Publication No. WO02/092756 which is assigned to the applicant of the present invention.

Telomerase Expression and Activity

A nucleic acid sequence comprising a DNA sequence encoding the RNA component of human telomerase is disclosed in U.S. Pat. No. 5,583,016. Further disclosed are a host cell transformed with the nucleic acid sequence and a method for producing the RNA component of human telomerase.

U.S. Pat. No. 5,629,154 discloses a method for determining telomerase activity in cells. The method comprising a step of placing an aliquot of a cell extract in a reaction mixture comprising a telomerase substrate lacking a telomeric repeat sequence and a buffer in which telomerase can catalyze extension of said telomerase substrate by addition of telomeric repeat sequences. A kit for detecting telomerase activity, comprising a telomerase substrate and a primer comprising a sequence complementary to a telomeric repeat sequence, is disclosed in U.S. Pat. No. 5,837,453.

U.S. Pat. No. 5,686,306 discloses a method for increasing the proliferative capacity of normal cells having telomerase activity. The method comprises culturing or cultivating the cells in the presence of an oligonucleotide substrate for telomerase under conditions such that the oligonucleotide substrate enters said cells and acts to lengthen telomeric DNA of said cells and the proliferative capacity of said cells is increased.

U.S. Pat. No. 5,891,639 discloses a method for measuring telomerase activity in a sample. The method comprising the steps of adding to a sample a telomerase substrate lacking a telomeric repeat sequence and a primer comprising a sequence sufficiently complementary to a telomeric repeat and after incubation in conditions that enable telomerase activity, correlating the presence of telomerase activity in the sample with the presence of molecules comprising an extended telomerase substrate bound to an extended primer.

Other methods for detecting and measuring telomerase activity are disclosed in U.S. Pat. Nos. 6,221,584; 6,221,590 and 6,489,097 among many others.

U.S. Pat. No. 6,475,789 discloses a mammalian cell that contains a recombinant polynucleotide comprising a nucleic acid sequence that encodes a telomerase reverse transcriptase protein, variant, or fragment having telomerase catalytic activity when complexed with a telomerase RNA.

Nowhere in the background art is it taught or suggested that selection for a subpopulation of cells that have retained endogenous telomerase activity may provide progenitor cells, which are partly committed to a given differentiated pathway, but which are not yet terminally differentiated.

### SUMMARY OF THE INVENTION

According to a first aspect the present invention provides partially committed telomerase positive progenitors derived from embryonic stem cells. Particularly, the present invention provides partially committed progenitors derived from embryonic stem cells, the progenitors expressing telomerase, and not being terminally differentiated and hence are capable of continued proliferation. More particularly, these telomerase-expressing progenitors that are not terminally differentiated and hence are capable of continued proliferation are useful for cell replacement therapy.

According to the present invention it is now disclosed that differentiation of embryonic stem cells stably expressing a fusion protein comprising a selection marker and a reporter gene under the control of the telomerase gene promoter can yield cell-lineage specific progenitor populations following exposure to selection pressure and/or through isolation via a traceable marker. Currently preferred selectable markers include but are not limited to antibiotic resistance genes, whereas currently preferred traceable markers are optically detectable markers including, but not limited to, fluorescent proteins.

We now disclose that unexpectedly telomerase promoter driven enhanced green fluorescent protein (EGFP) expressing human embryonic stem cells (HESC) can be detected among differentiated hESC in adherent conditions or in embryoid bodies (EBs), even 21 days after initiating differentiation and EB formation. Moreover, it appears that these progenitors express telomerase and are capable of proliferation and maintain active telomerase gene promoter.

According to another aspect of the present invention it is now disclosed for the first time that ectopic over-expression of telomerase gene in hESC does not adversely influence the differentiation capacity of these cells, and thus may enable the generation of a partially differentiated desired lineage of telomerase-expressing cells.

The cells of the present invention represent adult stem cells inasmuch as they are progenitors expressing telomerase that are capable of proliferation and maintain telomerase gene promoter activity, yet are committed to a particular differentiation pathway. Isolation and propagation of such subpopulations may overcome the problem of replicative arrest and/or senescence of lineage committed cells useful for cell replacement therapy.

According to another aspect of the present invention it is now disclosed that the persistence of telomerase promoter activity can be used to track or select these replication competent progenitor cells. Thus, for the first time it is possible to enrich or isolate adult stem cells, based on their endogenous telomerase activity as assessed by the persistence of telomerase promoter activity in these cells. Prolongation of the replicative capacity of committed progenitors serves as a valuable source of adult stem cells useful in cell replacement therapy. The telomerase-expressing progenitors selected according to the principles of the present invention may undergo terminal differentiation to mature cells of a particular cell lineage in vivo when used for cell replacement therapy.

According to an additional aspect of the invention, as a supplement or alternative embodiment to the foregoing, obtaining large number of hESC-derived cells that are useful for replacement therapy may be also achieved by additional procedures for genetic modification or immortalization of selected cell lines.

According to one embodiment, the present invention provides an enriched population of stem cells, comprising a plurality of committed progenitor cells stably transfected with a polynucleotide construct comprising a telomerase promoter active element operable linked to a sequence encoding a selectable marker.

According to certain embodiments the enriched stem cells are human stem cells; according to other embodiments the cells are non-human stem cells.

According to another embodiment of the present invention, human embryonic stem cells in culture are used to establish the appropriate stage(s) for intervention to preserve or to prevent the suppression of telomerase activity, which accompanies the differentiation of cell lineages.

According to yet another certain embodiment, the present invention provides an isolated subpopulation of stem cells, comprising a plurality of committed progenitor cells stably transfected with a polynucleotide construct comprising a telomerase promoter element operable linked to a sequence encoding a selectable marker.

According to yet another embodiment of the present invention, use of the human telomerase reverse transcriptase (hTERT) promoter fused upstream to a selectable marker or reporter gene, facilitates the enrichment and isolation of progenitors which retain proliferative capacity following commitment to a differentiation pathway.

According to yet an additional embodiment, the present invention provides a cloned population of stem cells, comprising a plurality of committed progenitor cells stably transfected with a polynucleotide construct comprising a telomerase promoter element operably linked to a sequence encoding a selectable marker.

According to yet an additional embodiment, the present invention provides a cloned population of stem cells, comprising a plurality of committed progenitor cells stably transfected with a polynucleotide construct comprising the hTERT gene or an active fragment thereof operably linked to a sequence encoding a promoter.

According to yet another embodiment of the present invention, stable over-expression of hTERT in pluripotent human embryonic stem cells is used for the generation of immortalized differentiated cell lineages.

In certain embodiments of the present invention, stem cells over-expressing ectopic telomerase can differentiate while maintaining higher telomerase activity than the telomerase activity of differentiated cells derived from non-transfected stem cells. This kind of genetic modification facilitates the generation of a large number of differentiated functionally relevant cells for replacement therapy.

According to yet another embodiment of the present invention, cell specific over-expression of hTERT can be used for extension of life span of enriched committed cell populations for cell replacement therapy.

### BRIEF DESCRIPTION OF THE DRAWINGS

The invention will be more fully understood and further advantages will become apparent when reference is made to the following detailed description of the invention and the accompanying drawings in which:

FIG. 1 presents the results from a TRAP assay for telomerase activity applied on extracts of hES cells at different stages of differentiation.

FIG. 2 demonstrates the expression of EGFP in undifferentiated hES cells transfected with pEGFP vector that express the EGFP reporter gene under the control of CMV promoter. Fluorescence was observed 48 hours after transfection (fluorescence inverted microscopy 20×).

FIGS. 3A-F show light fields and dark fields images of hESC clones stably expressing the EGFP gene under the control of the hTERT promoter, at different stages of differentiation.

FIG. 4A exhibits FACS analysis of stable hESC clones (G8 clone) that express the EGFP reporter gene under the regulation of the 5.9 Kb hTERT promoter fragment, grown in the non-differentiation conditions.

FIG. 4B exhibits FACS analysis of G8 clone following induction of differentiation.

FIG. 5 represents the results from a quantitative real time TRAP analysis of telomerase activity applied on extracts of HESC clones stably transfected with hTERT coding region driven by a constitutive β-actin promoter.

FIG. 6 is a scheme describing the selection process of progenitor cells expressing telomerase derived from hESC.

FIGS. 7A-B are schematic representations of the DNA constructs used for HESC transfection during the process of generating telomerase-expressing progenitor cells.

FIGS. 8A-B show a light field and a dark field images of hESC clones transfected with a vector comprising the construct depicted in FIG. 7A.

FIGS. 8C-D show a light field and a dark field images of HESC clone transfected with a vector comprising the construct depicted in FIG. 7B.

FIGS. 9A-B show a light field and a dark field images of hESC transfected with a vector comprising the construct depicted in FIG. 7A.

FIG. 10 is a scheme describing the process of generating insulin-producing cells from progenitor cells expressing telomerase derived from hESC.

FIGS. 11A-F show immunohistochemistry staining of embryoid bodies derived from hESC differentiation and of normal human pancreas, with an anti-insulin antibody.

### DETAILED DESCRIPTION OF THE INVENTION

1. Preferred Modes for Carrying Out the Invention

1.1 Definitions

The term “embryonic stem cells” or “ESC” refers to pluripotent cells derived from the inner cell mass of blastocysts with the capacity for unlimited proliferation in vitro in the undifferentiated state (Evans et al., Nature, 292:154-6, 1981). Embryonic stem cells can differentiate into any cell type in vivo (Bradley, et al., Nature 309: 255-6, 1984; Nagy, et al., Development, 110:815-821, 1990) and into a more limited variety of cells in vitro (Doetschman, et al., J. Embryol. Exp. Morph., 87: 27-45, 1985; Wobus, et al., Biomed. Biochim. Acta, 47:965-973, 1988; Robbins, et al., J. Biol. Chem., 265:11905-11909, 1990; Schmitt, et al., Genes and Development, 5: 728-740, 1991).

The term “adult stem cells” as used herein, refers to cells derived in vitro from human embryonic stem cells, and which like multipotential adult progenitor cells (also known as MAPC; e.g. Nature 418:41-9, 2002) have extended replicative capacity and a restricted differentiation capacity (partial lineage commitment).

As used herein, an “expression cassette” is a nucleic acid construct generated recombinantly or synthetically with a series of specified nucleic acid elements which permit transcription of a particular nucleic acid in a target cell. The expression cassette can be incorporated into a plasmid, chromosome, mitochondrial DNA, plastid DNA, virus, or nucleic acid fragment. Typically, the expression cassette portion of the expression vector includes, among other sequences, a nucleic acid to be transcribed, and a promoter.

As used herein “promoter” includes reference to a region of DNA upstream from the start of transcription and involved in recognition and binding of RNA polymerase and other proteins to initiate transcription. A promoter is capable of initiating transcription in cells. The promoter, according to the present invention, may be constitutive or non-constitutive. Tissue specific, cell type specific, and inducible promoters constitute the class of “non-constitutive” promoters. A “constitutive” promoter is a promoter which is active under most environmental conditions.

As used herein, the term “operably linked” refers to a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

The term “transfection” as used herein refers to the taking up of a vector by a host cell whether or not any coding sequences are in fact expressed. Numerous methods of transfection are known to the ordinarily skilled artisan, for example, CaPO₄ and electroporation. Successful transfection is generally recognized when any indication of the operation of this vector occurs within the host cell.

The term “senescence” as used herein refers to the loss of ability of a cell to replicate in the presence of normally appropriate mitogenic signals. Senescence is also typically accompanied by a change in expression patterns of one or more genes. For instance, senescence in some cells is accompanied by an increase in the expression of degradative enzymes, such as collagenase. The term senescence does not include quiescent cells that can be induced to re-enter the cell cycle under appropriate conditions. In most normal adult human somatic cells progressive rounds of cell division is associated with telomere length, ultimately reaching replicative senescence.

The term “telomerase reverse transcriptase” or “TERT” as used herein refers to a ribonucleoprotein enzyme with reverse transcriptase activity. Telomerase is capable of extending chromosome ends, i.e. “telomeres”, with a specific telomeric DNA sequence by using a portion of its RNA component as a template. The tern hTERT refers to a TERT of a human source. The terms hTERT and TERT may be used interchangeably.

A “TERT polynucleotide” as used herein, refers to a polynucleotide comprising a segment which is at least 85 percent identical to a naturally occurring TERT RNA sequence encoding the telomerase. Some TERT polynucleotides having sequence variations as compared to a naturally-occurring TERT sequence can be suitable as hybridization probes, PCR primers, LCR amplimers, and the like.

1.2 In Vitro Culture of ESC

The present invention provides embryonic stem cells capable of producing progenitors expressing telomerase, wherein said progenitors can proliferate and differentiate into a desired population of committed precursors or into fully differentiated cells while maintaining telomerase gene promoter activity.

Detailed procedures for culturing embryonic stem cells (e.g., ES-D3, ATCC# CCL-1934, ES-E14TG2a, ATCC# CCL-1821, American Type Culture Collection, Rockville, Md.). Embryonic stem cells display the following characteristics:
a. Normal diploid karyotype.
b. Capacity for indefinite propagation in the undifferentiated state when grown on a feeder layer.
c. Telomerase enzyme activity in the undifferentiated state.
d. Formation of multicellular aggregates, yielding outgrowths containing multiple identifiable differentiated cell types, including derivatives of the three major germ cell layers (ectoderm, mesoderm, endoderm) upon release from the feeder layer.

Embryonic stem cells display the innate property to differentiate spontaneously. In order to enrich the population of the undifferentiated ESC of the invention and to maintain its homogeneity, the innate spontaneous differentiation of these cells has to be suppressed. Methods for suppressing differentiation of embryonic cells may include culturing the undifferentiated embryonic cells on a feeder layer, such as of murine fibroblasts, also termed hereinafter “mouse embryonic fibroblasts” feeder layer or “MEFs”, or in media conditioned by certain cells.

A typical medium for isolation of embryonic stem cells may consist of 80% Dulbecco's modified Eagle's medium (DMEM; no pyruvate, high glucose formulation, Gibco BRL), with 20% fetal bovine serum (FBS; Hyclone), 0.1 mM β-mercaptoethanol (Sigma), 1% non-essential amino acid stock (Gibco BRL). FBS batches may be compared as it has been found that batches vary dramatically in their ability to support embryonic cell growth.

Alternatively, maintaining undifferentiated ESC in the laboratory may be achieved by the addition of a differentiation inhibitory factor (commonly referred to as leukemia inhibitory factor (or LIF) in the culture medium to prevent spontaneous differentiation (Williams, et al., Nature, 336: 684-687, 198S; Smith, et al., Nature, 336: 688-690, 1988; Gearing, et al, Biotechnology, 7: 1157-1161, 1989; Pease, et al., Dev. Biol., 141: 344-352, 1990). LIF is a secreted protein and can be provided by maintaining embryonic stem cells on a feeder layer of cells that produce LIF (Evans, et al., 1981; Robertson, Teratocarcinomas and Embryonic Stem Cells: A Practical Approach, Washington, D.C.: IRL Press, 1987) or by the addition of purified LIF (Williams, et al., 1988; Smith, et al., 1988; Gearing, et al., 1989; Pease, et al., Exp. Cell Res., 190: 209-211, 1990) to the medium in the absence of feeder layers. Differentiation of embryonic stem cells into a heterogeneous mixture of cells occurs spontaneously if LIF is removed, and can be induced further by manipulation of culture conditions (Doetschmann, et al., 1985; Wobus, et al., 1988; Robbins, et al., 1990; Schmitt, et al., 1991; Wiles, et al., Development, 111: 254-267, 1991; Gutierrez-Ramos, et al., Proc. Nat. Acad. Sci., 89: 9111-9175, 1992).

Embryonic stem cell differentiation can be variable between different established embryonic stem cell lines and even between laboratories using the same embryonic stem cell lines.

A method to produce an immortalized hESC population is disclosed in U.S. Pat. No. 6,110,739. The method comprising: (a) transforming an embryonic stein cell population with an immortalizing gene to create a transformed stem cell population; (b) culturing said transformed stem cell population under effective conditions to produce a transformed embryoid body cell population; and (c) incubating said transformed embryoid body cell population under conditions suitable to obtain an immortalized cell population that differentiates into cellular lineages comprising primitive erythroid cells and definitive erythroid cells. Methods for in vitro culturing of embryonic cell populations, utilizing combinations of growth factors for propagation and immortalization of these cells, are known in the art as for example disclosed in U.S. Pat. Nos. 5,690,926 and 6,110,739 and European Patent No. 380646, among many others.

An example for a purified preparation of pluripotent human embryonic stem cells is disclosed in U.S. Pat. No. 6,200,806. This preparation (i) will proliferate in an in vitro culture for over one year, (ii) maintains a karyotype in which the chromosomes are euploid and not altered through prolonged culture, (iii) maintains the potential to differentiate to derivatives of endoderm, mesoderm, and ectoderm tissues throughout the culture, and (iv) is inhibited from differentiation when cultured on a fibroblast feeder layer. The following cell surface markers characterize the purified preparation: SSEA-1 (−), SSEA-4 (+), TRA-1-60 (+), TRA-1-81 (+) and alkaline phospliatase (+).

Induction of differentiation in ES cells, preferably a controlled induction towards a specific cell lineage, is achieved for example by removing the differentiation-suppressing element, e.g. the feeder layer, from the culture. The embryonic stem cells may be placed in a culture vessel to which the cells do not adhere.

To effectively control the consequent differentiation, the cells must be in a homogeneous state. U.S. Pat. No. 6,432,711 provides a method for obtaining embryonic stem cells which are capable of differentiating uniformly into a specific and homogeneous cell line. The method comprises culturing embryonic stem cells under conditions which promote growth of the cells at an optimal growth rate. The embryonic stem cells then are cultured under conditions which promote the growth of the cells at a rate which is less than that of the optimal growth rate, and in the presence of an agent which promotes differentiation of the embryonic stem cells into the desired cell line. According to this method, a growth rate which is less than the optimal growth rate, is a growth rate from about 10% to about 80%, preferably from about 20% to about 50%, of the maximum growth rate for embryonic stem cells.

The growth rates for embryonic stem cells can be determined from the doubling times of the embryonic stem cells. In general, the optimum doubling time for embryonic stem cells is from about 13 hours to about 18 hours.

Any cell culture media that can support the growth and differentiation of embryonic stem cells, can be used with the present invention. Such cell culture media include, but are not limited to Basal Media Eagle, Dulbecco's Modified Eagle Medium, Iscove's Modified Dulbecco's Medium, McCoy's Medium, Minimum Essential Medium, F-10 Nutrient Mixtures, OPTI-MEM® Reduced-Serum Medium, RPMI Medium, and Macrophage-SFM Medium or combinations thereof. The culture medium can be supplied in either a concentrated (e.g.: 10×) or non-concentrated form, and may be supplied as either a liquid, a powder, or a lyophilizate. Culture media is commercially available from many sources, such as GIBCO BRL (MD, USA) and Sigma (MO, USA).

According to certain embodiment of the present invention, controlled differentiation in vitro of ES cells is conducted under serum-free conditions, also termed hereinafter knockout medium. Preferably, the knockout medium is enriched with supplements such as, serum replacements, nonessential amino acids, 2-mercaptoethanol, glutamine, growth factors e.g. human recombinant basic fibroblast growth factor (hrbFGF). In addition to the use of enrichment additives, the desired cell types may be further enriched and/or purified using selection markers and gene trapping based on the methods disclosed in U.S. Pat. No. 5,602,301.

For example, the embryonic stem cells may be placed in a culture vessel to which the cells do not adhere. Examples of non-adherent substrates include, but are not limited to, polystyrene and glass; The substrate may be untreated, or may be treated such that a negative charge is imparted to the cell culture surface. In addition, the cells may be plated in methylcellulose in culture media, or in normal culture media in hanging drops. Media which contains methylcellulose is viscous, and the embryonic stem cells cannot adhere to the dish. Instead, the cells remain isolated, and proliferate, and form aggregates.

1.3 Transient and Stable Transfections of ESC

ESC provide an in vitro tool to investigate at the cellular and molecular levels various developmental events, that cannot be studied directly in the intact human embryo, but which have important consequences to embryonic development. Like all normal diploid vertebrate cells ESC have a limited capacity to proliferate, a phenomenon that is known as replicative senescence or Hayflick limit.

Mechanisms to circumvent telomere attrition are necessary in those situations in which the extent of cell proliferation exceeds the ability to maintain a telomere length consistent with chromosome stability. In germline, this mechanism involves the expression of telomerase. Extension of telomere length to delay [or avoid] replicative senescence in culture is also important for the development of pluripotent hESC for therapeutic applications.

Telomere shortening and telomerase activity are involved in the processes of aging, cell senescence, and neoplastic transformation. Little is known about the dynamics of telomerase repression during human embryonic and fetal development. Telomerase activity is detectable at high levels in human blastocysts obtained from patients who had undergone in vitro fertilization, and in some human somatic tissues during early stages of prenatal development. Marked differences have been observed in the pattern of telomerase expression and timing of telomerase suppression among different fetal tissues due to the tissue-specific and developmental regulation of telomerase in the human fetus.

Telomerase was found to be active throughout the cell cycle in dividing immortal cells but its activity was repressed in quiescent cells that exit the cell cycle suggesting that loss of telomerase activity with differentiation appears to be a long-lasting state, even in differentiated cells that retain the ability to subsequently divide and do so ill the absence of telomerase activity (Holt et al., Proc. Natl. Acad. Sci. USA 94: 10687, 1997).

Persistent and ectopic telomerase expression was shown to overcome replicative senescence and immortalize certain differentiated somatic cells without interfering with differentiated cell function (e.g. Thomas et al., Nat. Biotechnol. 18: 39, 2000). A method for increasing the proliferative capacity of normal cells having telomerase activity by using exogenous telomerase substrate for lengthening of the telomeric DNA is disclosed in U.S. Pat. No. 5,686,306.

Sustained expression of TERT in mouse myocardial cell in the adult heart, caused a delay in ventricular myocytes exit from the cell cycle in the first month after birth and protection from cardiac myocyte apoptosis (Autexier et al., Trends Biochem. Sci 21: 387, 1996).

A variety of mechanisms are likely to be involved in the overall regulation of telomerase activity in different cell types wider various physiologic and pathophysiologic conditions. The protein (hTERT) and mRNA (hTER) components of the telomerase ribonucleoprotein, are each encoded at a separate genetic locus, and are under independent regulatory control. hTER has been reported in cells and tissues lacking detectable telomerase activity and telomerase activity most closely matches hTERT and not hTER expression profiles in various human fetal and adult tissues (Nakayama et al., Nat. Genet. 18: 65, 1998; Meyerson et al., Cell 90: 785, 1997). Taken together, these observations underscore the key role of hTERT mRNA expression as a regulator of overall enzymatic activity.

The hTERT gene spans 51 Kb with a genomic structure consisting of 16 exons and 15 introns, and is located in one copy on chromosome 5p15.33. Multiple length transcripts for the hTERT gene have been described, reflecting cell-specific alternative splicing. The 5′-flanking region of the hTERT gene has been characterized in part by sequence analysis, transient transfection assays of promoter-reporter constructs, as well as electrophoretic mobility shift assay (EMSA) and DNAse footprint analysis. These studies have identified a core promoter extending from −253 nt upstream of the transcription initiation site to 78 nt of exon 1. Several putative transcription factor binding elements within this core promoter sequence have been identified, including potential binding sites for Sp1, c-Myc, AP2, AP4, NF1 and a novel motif (MT-box), overlapping the location of the E-box adjacent to the translation initiation site of the gene (Tzukerman et al., Mol. Biol. Cell. 11: 4381, 2000).

An hTERT promoter as described herein can include any DNA sequence that is at least 85 percent homologous to a natural hTERT promoter or active fragments thereof, said DNA sequence is capable of being specifically bound by an RNA polymerase in such a manner that the RNA polymerase can unwind the DNA strand to initiate RNA synthesis of an hTERT gene.

In one embodiment of the present invention, genetic modification and selection may be used to enrich and isolate a subpopulation of cells by virtue of their property of retaining endogenous telomerase promoter activity.

Genetic modifications according to the invention are introduced to HESC by way of a vector comprising a synthetic polynucleotides encoding the desired molecules, for example, an active component of hTERT promoter operably linked to a reporter gene, a selection marker and the like. Introduction of synthetic polynucleotide into a target cell can involve one or more of non-viral and viral vectors, cationic liposomes, retroviruses, and adenoviruses such as, for example, described in Mulligan, R. C., (1993 Science 260:926). Vectors are employed with transcription, translation and/or post-translational signals, such as targeting signals, necessary for efficient expression of the genes in various host cells into which the vectors are introduced. Such vectors are constructed and transformed into host cells by methods well known in the art. See Sambrook et al., Molecular Cloning: A Laboratory Manual, Cold Spring Harbor (1989).

According to certain embodiment of the present invention hESC are transfected, in a transient or stable manner, with a vector that expresses a reporter gene wider the control of a promoter. The transient transfectants may also constitute the basis for selection of stable transfectants as exemplified hereinbelow.

In a certain embodiment of the present invention, non-differentiated HESC are transfected with pEGFP vector that expresses the EGFP reporter gene under the control of a CMV promoter.

In another certain preferred embodiment of the present invention, non-differentiated HESC are transfected with pEGFP vector that expresses the EGFP reporter gene under the control of an active fragments of the hTERT promoter.

Reporter genes that encode easily assayable marker polypeptides are well known in the art. In general, a reporter gene is a gene that is not present or expressed by the recipient organism or tissue and that encodes a polypeptide whose expression is manifested by some easily detectable property, e.g. phenotypic change or enzymatic activity and thus when co-transfected into recipient cells with a gene of interest, provide a means to detect transfection and other events. Among reporter genes appropriate to use according to the present invention, are those that encode fluorescent proteins. Of interest are fluorescent compounds and proteins, such as naturally fluorescent phycobiliproteins. Also are the fluorescent proteins that are present in a variety of marine invertebrates, such as the green and blue fluorescent proteins, particularly the green fluorescent protein (GFP) of Aequorea Victoria. The green fluorescent proteins constitute a class of chromoproteins found only among certain bioluminescent coelenterates. These accessory proteins are fluorescent and function as the ultimate bioluminescence emitter in these organisms by accepting energy from enzyme-bound, excited-state oxyluciferin (e.g., see Ward et al. in J. Biol. Chem. 254:781-8, 1979; Ward et al. Photochem. Photobiol. 27:389-96, 1978; Ward et al. Biochemistry 21:4535-40, 1982).

In another embodiment of the present invention, partially committed precursor or a partially differentiated cell lineage are transfected with an exogenous hTERT gene driven by a constitutive promoter, such as the powerful β-actin promoter or PGK gene promoter, and a selection marker. This approach is especially useful for promoting proliferation of specific cell lineages.

Numerous method for evaluating the effectiveness of transfection are know in the art. The effectiveness of transfection with a vector comprising an EGFP reporter gene may be monitored by straightforward fluorescence measurements as exemplified hereinbelow. The effectiveness of transfection with hTERT is preferably monitored by measurements of telomerase activity. Assays for measuring telomerase activity are known in the art, for example, Telomerase Repeat Amplification Protocol using TRAPeze® kit (Serologicals Corp., GA, USA) as exemplified hereinbelow.

1.4 Selection of Telomerase Active Progenitors

The present invention provides subpopulation of cells that are enriched and isolated by virtue of their property of retaining endogenous telomerase promoter activity.

According to one embodiment of the present invention, differentiated derivatives of hESC stably transfected with the gene expressing the catalytic component of telomerase (hTERT) may be generated by selection, wherein the selected cells retain high levels of telomerase activity, even following differentiation.

A strategy for selection of telomerase-expressing progenitors population may comprise the following elements by a way of non-limiting example:
1. transfection of HESC with a construct that carries a gene encoding a fusion protein of a resistance gene and reporter gene under the control of hTERT promoter or active fragments thereof; and
2. selection of resistant hESC clones; and
3. transfer of resistant clones to differentiation growth conditions; and
4. characterization of the differentiation potential of the new adult stem cells population; and
5. characterization of the growth potential of the new adult stem cells population; and
6. selection of desired lineage specific progenitors from the new adult stem cells population.

According to a preferred embodiment of the present invention, progenitors expressing telomerase can be identified and isolated by tracking the endogenous telomerase promoter activity, to provide cell subpopulations useful for cell replacement therapy. Various means for tracking promoter activity are known in the art. Promoter activity can be determined by measuring the difference upon stimulation in mRNA transcribed by genes under the control of the promoter. Alternatively, the level of protein produced from this transcribed RNA can be determined before and after stimulation. For example, promoter activity can be measured in a quantitative northern blot which directly measures the amount of mRNA in a selected sample which is transcribed from a gene regulated by the promoter. Performing quantitative northern blot analysis is well known in the art. Similarly, the level of RNA can be measured indirectly using quantitative RT-PCR. Measuring the level of protein encoded by an RNA is particularly suitable for proteins which are not translationally regulated, so that the level of protein corresponds to the amount of RNA which is transcribed from a gene under the control of a promoter. Protein determinations are routine in the art, commonly being performed by western blot analysis, ELISA or other affinity detection techniques which monitor the level of protein in a sample (see generally, Current Protocols in Molecular Biology, F. M. Ausubel et al., eds., Greene Publishing Associates, Inc. and John Wiley & Sons, Inc., (1995 Supplement), Coligan, CURRENT PROTOCOLS IN IMMUNOLOGY, Wiley/Greene, N.Y. (1991); and Harlow & Lane, ANTIBODIES: A LABORATORY MANUAL, Cold Spring Harbor Press, N.Y. (1989)). The level of induction of a promoter by an appropriate stimulus by such methods refers to the percent or fold increase in the production of transcribed or translated gene products under the control of a promoter in response to the stimulus.

In a preferred embodiment of the present invention the activity of hTERT promoter is monitored by means of a reporter gene. Examples of reporter genes suitable for use in the present invention are given hereinabove.

Promoter-reporter systems that may be used in the present invention include vectors, such as the pSEAP-Basic, pSEAP-Enhancer, pβgal-Basic, pβgal-Enhancer, or pEGFP-1 Promoter Reporter vectors available from Clontech (BD Biosciences Clontech, CA, USA). Each of these promoter-reporter vectors include multiple cloning sites positioned upstream of a reporter gene encoding a readily assayable protein such as secreted alkaline phosphatase, β-galactosidase, or green fluorescent protein. Upon stimulation, the level of reporter protein is assayed and compared to appropriate control systems. If necessary, the sequence of an enhancer can be added to the promoter-reporter system for augmenting transcription levels from weak promoter sequences. A promoter may be also linked to a retrovirus-based luciferase reporter-gene system. The luciferase activity obtained upon stimulation can be compared to the level using a promoterless construct to obtain a measure of relative promoter activity. Currently preferred promoter-reporter systems are hTERT core promoter with EGFP reporter gene coding region or the fusion protein of histone H₂A and GFP DNA encoding region, as exemplified hereinbelow.

In certain preferred embodiment of the present invention, HESC clones are stably transfected with hTERT promoter fused to a selectable marker, preferably a drug resistance gene (e.g., ampicillin resistance gene, hygromycin resistance gene, GENETICIN™ (G418; BD Biosciences Clontech, CA, USA), mitomycin resistance gene, kanamycin resistance gene, neomycin resistance gene etc.) and a reporter gene, to enable selection for telomerase-expressing populations of progenitors following selection pressure. By way of one non-limiting example, hTERT promoter-neomycin/EGFP reporter construct, can be used to obtain stable transfectants expressing endogenous hTERT. An advantage of using neomycin for the purpose of selection is the convenient availability of G418 (BD Biosciences Clontech, CA, USA) resistant mouse colonies that enable preparation of G418 resistant mouse embryonic fibroblasts (MEFs).

Based on the results exemplified hereinbelow showing that EGFP positive cells are detectable among differentiated HESC in adherent conditions or in EBs, even 21 days after initiating differentiation and generating EBs, it appears that EGFP positive cells of the present invention represent telomerase-expressing progenitors which are capable of proliferation and maintain active telomerase gene promoter.

During the process of differentiation of the pluripotent hESC into various cell derivatives, progenitors committed to specific cell lineages are generated. Proliferation of the selected EGFP positive progenitors of the present invention can proceed through two different paths producing two types of populations: (a) a downstream path towards differentiation, resulting in mature cell-lineage specific cells that most likely do not express telomerase; and (b) a horizontal path of proliferation and maintenance of cell-lineage specific progenitor populations that maintain an active telomerase gene.

Selection procedure for specific progenitor populations, according to the horizontal path of proliferation, is achieved by exposure to an additional selection pressure that is generated upon the addition of a lethal substance. Particularly, differentiation of hESC of the present invention, which are stably expressing a fusion reporter gene consisting of the neomycin selection marker and EGFP under the control of the telomerase gene promoter, can yield cell-lineage specific progenitor populations following exposure to neomycin.

By means of non-limiting example, addition of G418 to the growth medium of hESC expressing telomerase, transfected with an hTERT promoter-neomycin/EGFP reporter construct, at the estimated suitable day for selection yields population of specific progenitor populations that maintain telomerase promoter activity following differentiation. The surviving cells may be progenitors or adult stem cells that should be OCT-4, SSEA-3, SSEA-4, TRA-1 and alkaline phosphatase negative, however these cells express telomerase. It is possible that a residual portion of the surviving cell population may be of undifferentiated cells. The surviving cells may be characterized by methods known to persons skilled in the art, in terms of lineage markers, growth potential and other cellular and cell-line characteristics. It is also possible to distinguish between two different populations of the surviving cells that is a population of differentiating cells and a population of cells in a growth arrest phase.

1.5 Selection of Cell Lineages Derived from hESC Expressing Telomerase

Induction of differentiation of HESC clones required differentiation growth conditions selected from:
a. Adherent conditions—without MEFs and in the presence of a selection drug.
b. Suspension, to induce differentiation into EBs for 7 days, and then dissociate and plate the EBs in the presence of a selectable marker.

According to certain preferred embodiment of the present invention, differentiation of HESC clones is induced in suspension. The suspension protocol is based on the postulate that progenitors cell population have to pass the stage of EBs formation and creation of three germ layers. Following induction of differentiation residual undifferentiated hESC may be present in the overall population of cells expressing telomerase, however such residual cells are expected to eventually differentiate due to the absence of a feeder layer.

According to an additional preferred embodiment of the present invention, hTERT-expressing differentiated cell lineages may be generated by stable transfections of a partially committed precursor or of a partially differentiated cell lineage with hTERT gene coding sequence driven by a powerful promoter, such as the β-actin gene promoter or PGK gene promoter, and with a selection marker. This strategy enables to overcome the disadvantage of low proliferation capacity and limited life span and to promote proliferation of specific cell lineages. The final clone may be examined for the criteria which characterize the desired cell line and for telomerase activity, telomere length, extension of life span and lack of tumorigenic properties as outlined above.

A desired lineage specific progenitors may be selected either by using specific differentiation growth conditions or by transfection with cell specific promoter driving a second selection.

Many standard means to determine the presence of a more differentiated cell are well known in the art. For example, RT-PCR applied to RNA extracted from differentiated hES cells enables detection of a variety of differentiated cell markers.

According to one embodiment of the present invention, hTERT over-expressing hES cells are allowed to differentiate under specific conditions chosen for selection and enrichment for production of the selected cell lineage that it is desired to obtain. As is commonly known in the art these conditions include the use of varied cell growth factors, growth supplements, antioxidants or any other selected modifications to the culture medium that are known to predispose the cells to commit to a particular cell lineage.

According to one embodiment of the present invention, a committed cell lineage is obtained by introducing to human ES cells a cell specific promoter fused to an antibiotic resistance gene or any other selectable marker. By way of non-limitative example, a cell specific promoter-neo^{r} transgene is utilized in a way that permits the generation of homogeneous selected cell lineages, in the context of an appropriate (e.g. bicistronic) expression vector that contains an attenuated internal ribosome entry site (IRES). This vector is constructed so that hTERT coding sequence is driven by a cell specific gene promoter that becomes active in cells committed to the desired cell lineage very early stages the differentiation pathway.

According to another embodiment of the present invention, the hTERT coding region is constructed to reside in a single expression cassette with an IRES and a first antibiotic resistance selection marker. A currently preferred marker for this construct is Neomycin. The construct is transfected into undifferentiated hES cells and resistant clones are selected, following differentiation. Following this selection protocol, all the transfected cells that express the selection marker also express telomerase and differentiate into the desired cell lineage that has the ability to activate the cell-specific promoter at the very early stages of this differentiation pathway.

According to another embodiment of the present invention, during the selection process for specific cell lineages cells are examined for advantageous properties and/or lack of deleterious properties, as follows:

(i) Expression of the Neo^{r} Gene at the Undifferentiated State.

This examination may be performed using RT-PCR with appropriate primers for detection of differentiation-specific sequences. This step is important as it facilitates to avoid proceeding the selection procedure with false positive clones.

(ii) Expression of Exogenous hTERT or Other Proliferation (Extended Life-Span) Promoting Gene.

This indication is measured in clones selected after differentiation and first antibiotic selection (e.g. hygromycin). For this type of measurements quantitative methods are applied, for example, northern blot analysis, RT-PCR, TRAP assay for telomerase activity, TRF assay for telomere length. The above indication is evaluated at multiple time intervals following differentiation. As control, normal (non-transfected) HESC that have gone through the same process are used.

(iii) Expression Profile of Factors or Activities Involved in the Desired Differentiation Pathway.

The new sub-clones are further characterized for typical activity or cellular marker that is expressed in the cell lineage of choice. The induction of expression of new or increased levels of proteins such as enzymes, receptors and other cell surface molecules, amino acids, peptides and other bioactive molecules, can be analyzed with any technique known in the art which can identify the alteration of the level of such molecules. These techniques include immunohistochemistry using antibodies against such molecules, or biochemical analysis. Such biochemical analysis includes protein assays, enzymatic assays, receptor binding assays, enzyme-linked immunosorbant assays (ELISA), electrophoretic analysis, analysis with high performance liquid chromatography (HPLC), Western blots, and radio-immuno-assays (RIA). Nucleic acid analysis such as Northern blots can be used to examine the levels of mRNA coding for these molecules, or for enzymes which synthesize these molecules. The preferred method is quantitative RT-PCR. The pattern of expression is compared with that of control cells. A microchip for expression profile may also be used.

(iv) Quality Control of the Differentiated Cells Derived as Above from hES Cells.

This type of measurement is applied using appropriate parameters, including but not limited to ultrastructural characterization using methods such as electron microscopy, electrophysiological profile, metabolic profile, or any other suitable parameter for testing the selected cells.

(v) Tumorigenic Properties.

Cell lacking tumorigenic properties are selected according to any of the criteria as are known in the art, including the inability to generate tumors in nude mice, the inability to grow on soft agar (focus formation), and presence of a normal cell karyotype.

2. Therapeutic and Research Applications of hTERT-Expressing Progenitor Cells

ESC provide an in vitro tool to investigate at the cellular and molecular levels various developmental events that cannot be studied directly in the intact human embryo but which have important consequences to embryonic development.

ES cells can be used to screen for factors which produce ES derivative (more differentiated) cells as different combinations of growth factors in the culture medium are known to result in distinct patterns of cell lineage differentiation.

These progenitors cell population of the present invention can be used as a system to examine telomerase gene regulation at the promoter level and to ascertain how these cells maintain telomerase activity.

ES cells confer a therapeutic potential as they may serve as a useful source of cells for transplantation and cell therapy upon differentiation into a desired cell population. A method for treating a human subject by administering a therapeutically effective amount of human mesenchymal stem cells, is disclosed in U.S. Pat. No. 6,355,239. The stem cells according to this patent, may express incorporated genetic material of interest.

The present invention discloses functional hESC progenitors suitable for the purpose of cell therapy as well as strategy and methods for preparing thereof. The method of the present invention for selection of hES derived cell, relates to a positive selection scheme. Thus, a marker gene, such as a gene conferring antibiotic resistance (e.g. neomycin, hygromycin), is introduced into the stem cells under appropriate control such that expression of the gene occurs only in the desired cell lineage. For example, the marker gene can be under the control of a promoter which is active only in the desired cell linage. Upon differentiation of the stem cells, the desired lineage is then selected based upon the marker, e.g. by contacting the mixed cells with the appropriate antibiotic to which the desired lineage has been conferred resistance. Cell line other than the desired line will thus be killed, and substantially pure, homogeneous population of the desired line can be recovered. In more preferred methods, two markers are introduced into the parent stem cells, one allowing selection of vector-transfected stem cells from non-transfected cells, and one allowing selection of the desired cell lineage from other lineages. A double positive selection scheme can thus be used where each selectable marker confers antibiotic resistance. Using this selection methodology greatly enriches the population of the desired cell linage.

According to one embodiment of the present invention, HESC stable clones expressing EGFP under the control of the insulin promoter may provide a potential source for generating IPC for cell replacement therapy.

HES cells of the present invention may be suitable for implantation into individuals in need thereof. The cells can be introduced in any suitable manner, but it is preferred that the mode of introduction be as non-invasive as possible. Thus, delivery of the cells by injection, catheterization or similar means will be more desired.

2.1 Cell Replacement Therapy for Treating Diabetes Mellitus

Diabetes Mellitus (DM) is a heterogeneous metabolic disease caused by absolute (type I) or relative (type II) insufficiency of the capacity of pancreatic β-cells to produce insulin in amounts sufficient to meet the body's needs. Resulting sustained hyperglycemia is a major contributor to several complications including cardiovascular disorders, kidney failure and blindness. Type I diabetes is an autoimmune disease that usually begins in childhood or early adulthood and eventually causes complete destruction of the insulin secreting β-cells in the pancreas. Type II non-insulin dependent diabetes mellitus (NIDDM) results from resistance of peripheral tissues to insulin action, producing a progressive state of relative insulin deficiency that is treated primarily with medications, sometimes supplemented with insulin replacement as well. Studies of islet and pancreatic transplantation have shown that prevention of diabetes complications can be achieved by the level of physiologic glucose control affected by authentic β-cell function. This conclusion was further highlighted by the successful implementation of an islet transplantation protocol using a glucocorticoid-free anti-rejection regimen (Soria et al., Nat. Biotech. 18: 399, 2000). However it is quite obvious that the availability of human islets will always be a limitation in meeting public health needs in diabetes treatment. As a result, much effort has been expended in developing alternative sources of physiologically regulated insulin producing cells (IPC). The potential of β-cell lines derived from rodents as source for cell replacement therapy is known in the art along with approaches involving extending the β-cells phenotype to other tissues using ill vivo gene transfer either by expressing the insulin gene or an insulin gene analogue under the control of a glucose sensitive promoter or by ectopic expression of Ipf1/Pdx1.

Human embryonic stem cells provide a potential source for insulin producing cells (IPC) replacement therapy. It has already been shown that mouse embryonic stem cells (mESC) can be engineered to allow selection for cells that were differentiated into IPC (Halvorsen et al., J. Endo. 166:103, 2000). A protocol was established for inducing differentiation of mESC into IPC that responded to normal glucose concentrations by secreting low levels of insulin into the growth medium (Klug et al., J Clin Invest. 98:216, 1996). In a recent study it was proposed that hESC can be manipulated in culture to express the Pdx1 gene that regulates insulin transcription in β-cells (Ofir et al, supra). Pending International Publication No. WO02/092756 discloses hESC which under appropriate culture conditions differentiate into a substantially high percentage of insulin producing cells in spontaneously formed aggregated embryoid bodies and show positive immunohistochemical staining with anti-insulin antibodies. These clusters of cells that express genes characteristic of pancreatic p-cells function such as ngn3, Pdx1, Glut-2 and islet specific glucokinase also secrete insulin into the medium.

Nevertheless, elucidation of the mechanisms underling the repression of telomerase activity during cellular differentiation, raises important issues at the practical level, since scaling up will be required for obtaining large number of HESC derived IPC for replacement therapy. Given that telomerase is markedly reduced in terminally differentiated cells eventually leading to cellular senescence, it is disclosed in the present invention that ectopic expression of hTERT in undifferentiated hESC may overcome the limited number of cell divisions of differentiated cells derived upon induction of hTERT over-expressing HESC differentiation. This kind of genetic modification may facilitate obtaining a large numbers of differentiated functionally relevant cells for replacement therapy. Accordingly it is important to understand the regulatory mechanisms for hTERT repression and to determine whether or not ectopic expression of hTERT in hESC before differentiation, for purposes of subsequent constitutive expression and immortalization, interferes with derivation of differentiated cell types, or renders the derivative cells more prone to malignant transformation. The interplay between telomerase regulation, immortalization, and differentiation is central to the successful application of this promising approach. Of note, retroviral transduction of the hTERT gene did not prevent the senescence phenotype of human β-cell-enriched islet cultures (Halvorsen et al. J. Endo. 166: 103, 2000), most likely since the cells have already adopted a telomere-independent senescence program that could no longer be circumvented by hTERT ectopic expression.

In a preferred embodiment of the present invention, a strategy is provided for generating fully differentiated IPC using ectopic expression of hTERT at the undifferentiated stage, through a controlled differentiation process.

2.2 Generating Models of Specific Human Genetic Diseases

The present invention provides methods that may be used for generating transgenic non-human primates for models of specific human genetic diseases. Such application requires selection and isolation of primate embryonic stem cells, as taught by the present invention or as disclosed for example in U.S. Pat. No. 6,200,806, further transfected with a desired gene which will allow the generation of primate tissue or animal models for any human genetic disease for which that gene has been cloned and identified as responsible for said disease. Such animal model is essential for elucidating mechanisms of disease and for testing new therapies.

The following examples are to be construed in a non-limitative fashion and are intended merely to be illustrative of the principles of the invention disclosed.

### EXAMPLES

### Example 1

### In Vitro Culture of Embryonic Stem Cell

Large stocks of primary mouse embryonic fibroblasts (MEFs) were prepared as described by Robertson (Robertson E. G. Ed., Teratocarcinomas and embryonic stem cells: a practical approach in Practical approach series, IRL Press 1987, 71-112) and stored in liquid nitrogen. After each thaw, cells were used for only 3-5 passages.

The ES H9 cells were maintained in the undifferentiated state by propagation in culture on a feeder layer of MEFs that have been mitotically inactivated by γ-irradiation with 35 Gy and plated on gelatin coated six-well plates. Cells were grown in knockout DMEM (GIBCO/BRL, Grand Island, N.Y.) supplemented with 20% serum replacement (GIBCO/BRL), 1% nonessential amino acids (GIBCO/BRL), 0.1 mM 2-mercaptoethanol (GIBCO/BRL), 1 mM glutamine (Biological Industries, Ashrat, Israel), 4 ng/ml human recombinant basic fibroblast growth factor (hrbFGF, PeproTech Inc, Rocky Hill, N.J.). Cultures were grown in 5% CO₂, 95% humidity and were routinely passaged every 4-5 days after disaggregation with 0.1% collagenase IV (GIBCO/BRL).

Differentiation of ESC was induced using methods described in Robertson et al. (supra) and Keller (Curr. Op. Cell Biol. 7:862, 1995). In brief, about 107 undifferentiated ES cells were disaggregated and cultured in suspension in 100 mm bacterial grade petri dishes (Greiner, Frickenlausen, Germany), which results in induction of synchronous differentiation, characterized by initial formation of small aggregates, followed by the acquisition of the configuration of embryoid bodies (Itskovitz-Eldor et al., 2000). Alternatively, ES colonies were left unpassaged until confluence (about 10 days), and then were replated on gelatinized six-well tissue culture plates in the absence of a feeder layer. The cells spontaneously differentiated to an array of cell phenotypes. The growth media that were used in differentiation were as described above.

### Example 2

### Telomerase Activity During hESC-Differentiation

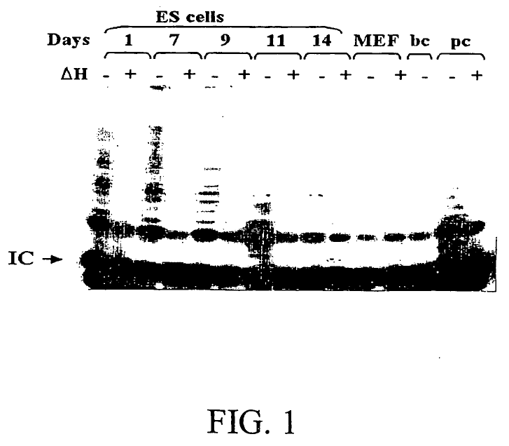

Telomerase Repeat Amplification Protocol (TRAP) is a highly sensitive polymerase chain reaction (PCR)-based assay for measuring telomerase activity. TRAP assay was performed using extracts from hESC cultured on MEF cells (FIG. 1, +ΔH) and from hES cells that were allowed to differentiate for 7, 9, 11 and 14 days (FIG. 1, −ΔH). As controls, TRAP activity was measured in extracts of MEF [FIG. 1, buffer control (bc) lane], and in extracts of cells expressing telomerase supplied in the kit (FIG. 1, pc lane; Roche Diagnostics Corporation, IN, USA). The results shown were obtained using 5 μg of each extract without and with 15 minutes at 85° C. heat inactivation. A 36 bp internal control for amplification efficiency and quantitative analysis was used for each reaction as indicated by the arrowhead (FIG. 1, IC band).

Measurement of telomerase activity using the TRAP assay demonstrated that HESC express high levels of telomerase and that spontaneous differentiation of these cells is associated with a striking and time-dependent decline in telomerase activity by day 14 (FIG. 1). RT-PCR measurements confirmed that suppression of telomerase activity with differentiation occurs at the level of the endogenous telomerase mRNA.

### Example 3

### Transient and Stable Transfections of hES Cells

Undifferentiated hES cells were transfected with an enhanced green fluorescent protein (EGFP) vector (pEGFP) that expresses the EGFP reporter gene under the control of CMV promoter. Fluorescence was measured 48 hours after transfection.

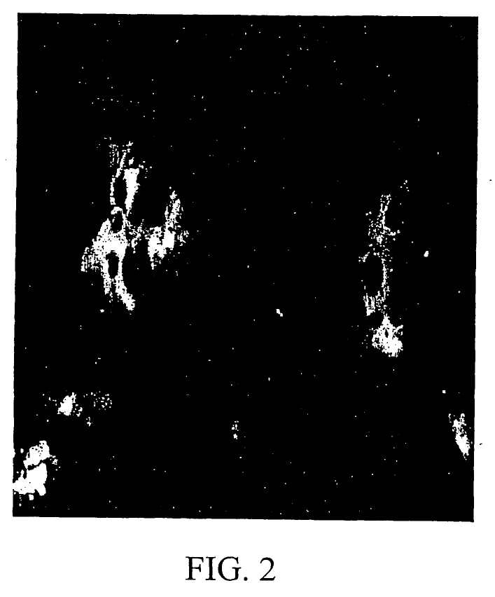

Multiple protocols were tested for both transient and stable transfection of undifferentiated hES cells, using pEGFP-Cl (BD Biosciences Clontech, CA, U.S.), with the CMV promoter fused upstream of the EGFP reporter gene. An appropriate ratio of plasmid DNA to the commercial FuGENE™ 6 transfection reagent (Roche Applied Science, IN, U.S.) was found to yield transient transfection with approximately 30% efficiency, as evident by percentage of cells displaying green fluorescence (FIG. 2; fluorescence inverted microscopy 20×). At G418 concentration of 200 μg/ml, colonies of GFP positive cells have survived and expanded, constituting the basis for selection of stable transfectants in the further experiments described below.

### Example 4

### hTERT Promoter Transcriptional Activity in hESC

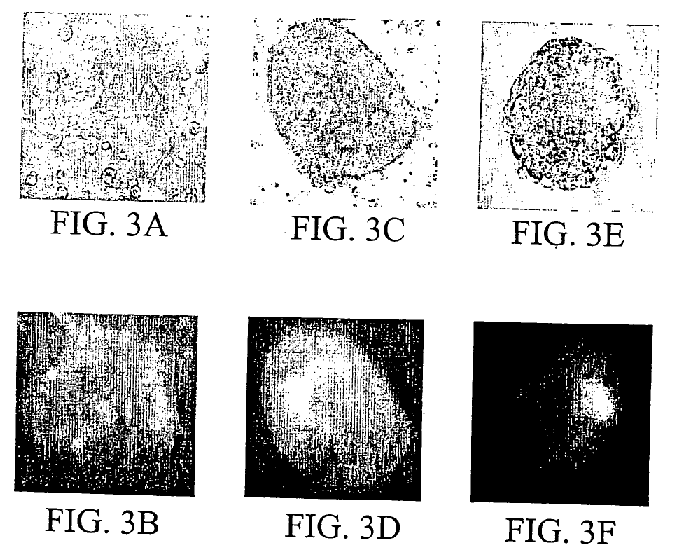

Transient transfection assays were used to examine the transcriptional activity of the hTERT promoter in HESC. Three promoter subfragments of different sizes were fused upstream to a luciferase reporter gene, as was previously described⁵¹, demonstrated high levels of transcriptional activation in all cells (FIGS. 3A-B). Of note, the smallest promoter fragment of 283 bp (core promoter), is sufficient for yielding maximum promoter activity in HESC as have been observed in other cell lines (Ofir et al., Proc. Natl. Acad. Sci. USA 96:11434).

Following differentiation and EB formation, a non-homogenous reduction in EGFP intensity is observed at 4 days (FIGS. 3C-D) and at 18 days (FIGS. 3E-F).

### Example 5

### hESC Clones Stably Transfected with the hTERT Promoter/EGFP Reporter Construct

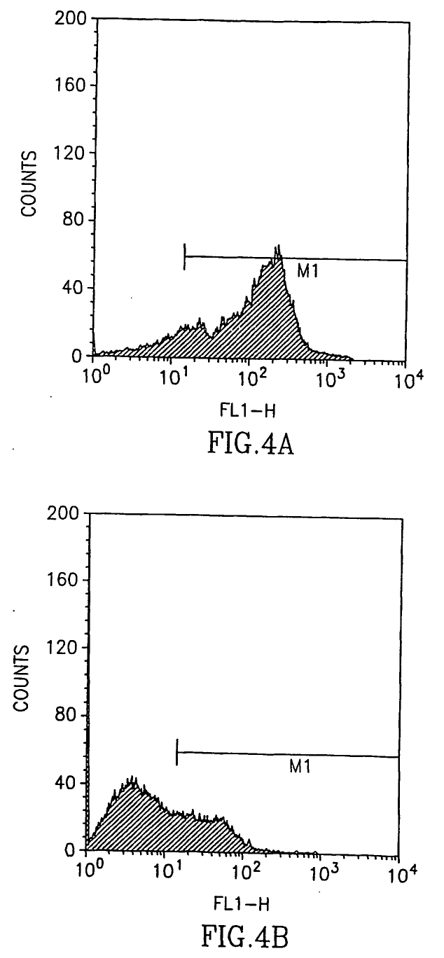

In order to examine the regulation of the hTERT gene promoter during the differentiation of different cell lineages, we have generated stable hESC clones (G8 clone) that express the EGFP reporter gene under the regulation of the 5.9 Kb hTERT promoter fragment. As expected, undifferentiated HESC colonies displayed positive EGFP signals in all cells (FIG. 4A). In vitro differentiation of these HESC clones in suspension resulted in formation of aggregated embryoid bodies, displaying diffuse pattern of EGFP expression. Following transition to differentiation conditions, a non-homogeneous reduction in EGFP intensity was observed at day 4 and at day 18 presumably reflecting differential suppression of hTERT promoter activity (FIG. 4B). FACS analysis revealed that only 30% of the cells remain EGFP positive after a week of growth in differentiation conditions.

### Example 6

### Stable hESC Clones Ectopically Expressing the hTERT Gene

To examine the effect of exogenous hTERT over-expression on HESC differentiation, immortalization and tumorigenesis, we have generated hESC clones stably transfected with the hTERT cDNA coding region driven by a constitutive β-actin promoter.

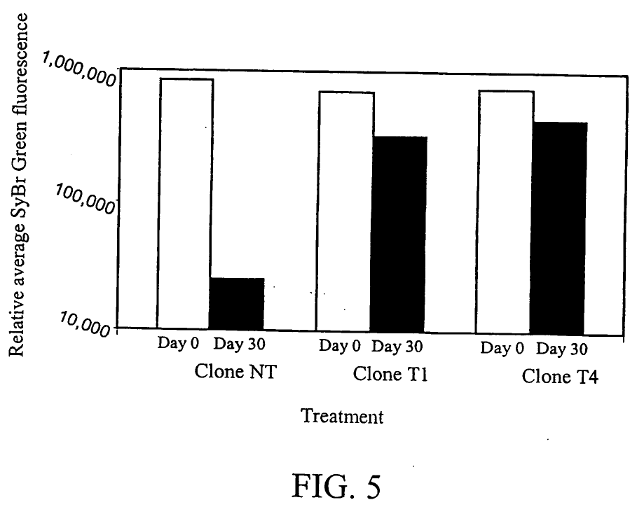

Out of several clones derived from this selection process, we examined telomerase activity in two clones (FIG. 5; T1 and T4 clones) in the undifferentiated state (FIG. 5; Day 0) and following transition to differentiation growth conditions (FIG. 5; 30 Day) compared to a clone of non transfected HESC (FIG. 5; clone NT). The expression of the exogenous hTERT mRNA was examined by a quantitative TRAP (Q-TRAP) assay developed in our laboratory that uses the TRAPeze® telomerase detection kit (Serologicals Corp., GA, USA) with our own modification and adaptation for real-time amplification on the Rotor-Gene real time PCR apparatus (Corbett Research, NSW, AUSTRALIA). Logarithmic values of the relative telomerase activity were determined from the relative averaged SYBR® Green Fluorescence (Applied Biosystems, CA, U.S.).

The results obtained revealed high telomerase activity in undifferentiated non-transfected hESC (FIG. 5; NT) as well as in the two stable transfected clones (FIG. 5; T1 and T4 clones). Following transition to differentiation conditions, telomerase activity decreased in non-transfected hESC but high levels persisted in the stable clones (FIG. 5).

### Example 7

### Selection for Progenitors Expressing Telomerase

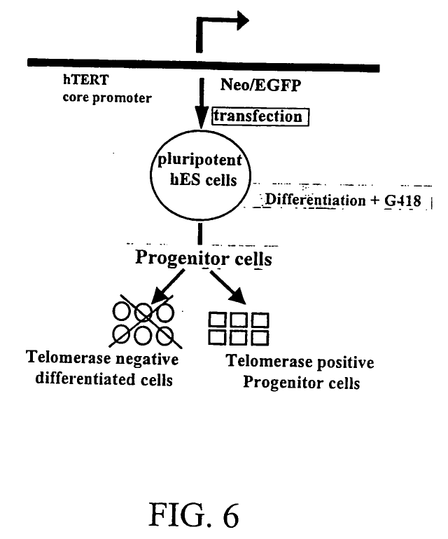

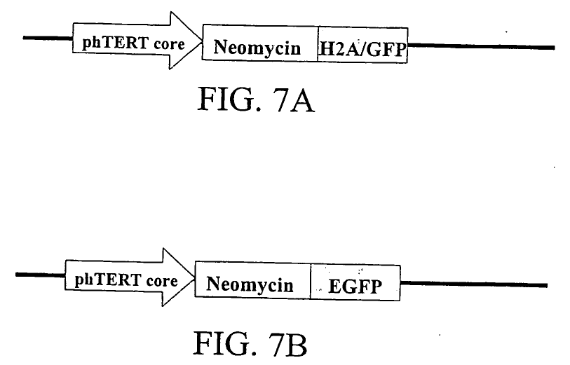

A strategy for selection of a population of progenitor cells expressing telomerase is schematically described in FIG. 6. For generating progenitor cells that express telomerase, two DNA constructs were prepared comprising the hTERT core promoter-selection marker/reporter gene including the following polynucleotide sequences: neomycin resistance gene coding region, EGFP reporter gene coding region and DNA encoding the fusion protein of histone H₂A and GFP. Neomycin gene was ligated to EGFP in-frame resulting in the neo-EGFP cassette, and to H₂A-GFP in frame resulting in the H₂A-GFP cassette. Both cassettes, neo-EGFP (FIG. 7A) and neo-H₂A-GFP (FIG. 7B), were ligated to the hTERT core promoter in the background of pGL₃-basic in which the luciferase gene was deleted (FIGS. 7A-B).

The GFP reporter gene was deliberately located at the end of each cassette to monitor transcription efficiency. The EGFP gene is expressed in the cytoplasm and the H₂A-GFP is bound to the chromatin and hence the reporter glow is observed in cells nuclei.

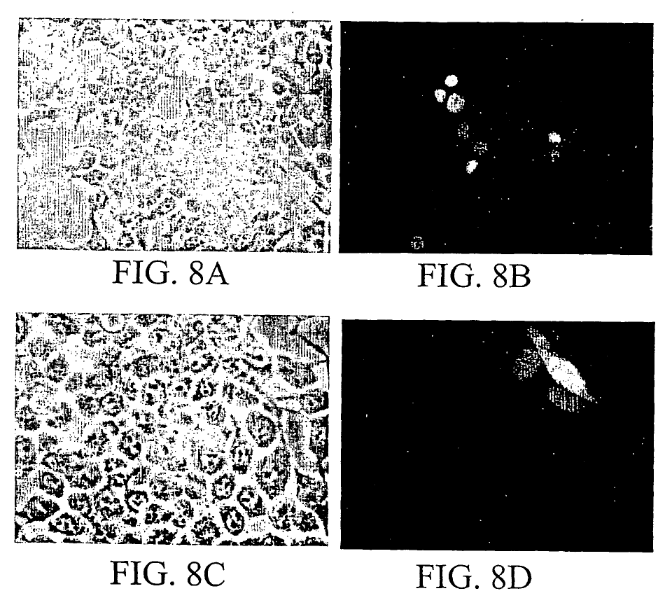

Human embryonic kidney cells expressing telomerase (HEK293; ATCC) were transiently transfected with vectors comprising one of the constructs. As anticipated, the expression of H₂A-GFP was observed in the nuclei of the cells (FIGS. 8A and 8B) and the expression of EGFP was observed in the cytoplasm of the cells (FIGS. 8C and 8D).

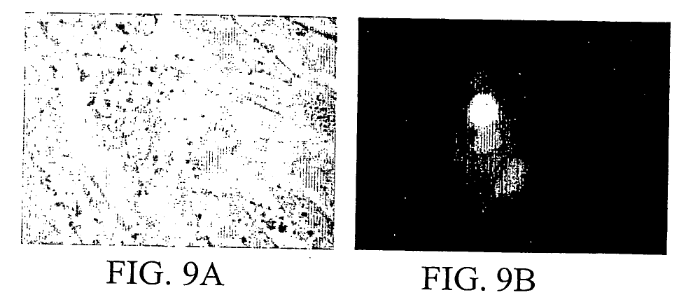

Human embryonic stem cells were transfected with the constructs using several transfection reagents including FuGENE™ 6 (transfection with FuGENE™ 6 is demonstrated in FIG. 1.). Transfection using the cationic polymer transfection reagent jetPEI™ (Qbiogene, Inc., CA, USA) provided the highest transfection efficiency, as exemplified in FIG. 9 for HEK293 cells transfected with a vector comprising the H₂A-GFP cassette ligated to the hTERT core promoter. Neomycin resistant clones are subjected to G418 selection.

### Example 8

### Insulin Production in Embryoid Bodies Derived from hESC Differentiation

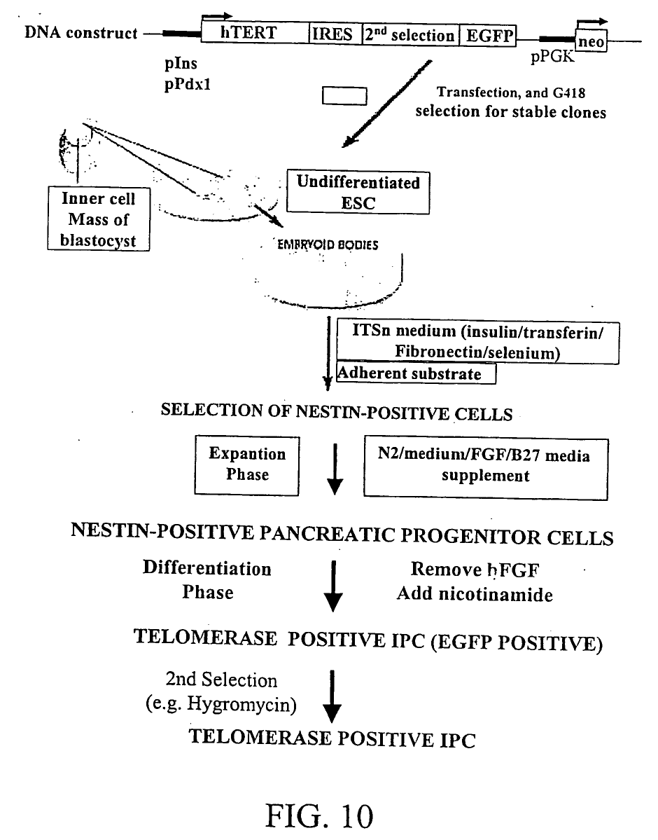

Insulin producing cells (IPC) are generated from hESC transfected with hTERT promoter according to the strategy described in FIG. 10. Initially, HESC are stably transfected with pEGFP-1 vector (BD Biosciences Clontech, CA, USA) that carries the EGFP reporter gene fused downstream to the insulin minimal promoter (5′ flanking region; −327 bp to +30 bp of the human insulin gene; Ofir et al., supra). Stable HESC clones are selected using G418 and the expression of the neomycin resistance gene according to FIG. 10. Following growth under differentiating conditions, expansion and a second selection process, the desired IPC clones are generated. The following methods may be applied for evaluating insulin production in cells generated by this strategy: immunohistochemistry, Reverse Transcriptase-Polymerase Chain Reaction (RT-PCR) and EGFP gene expression.

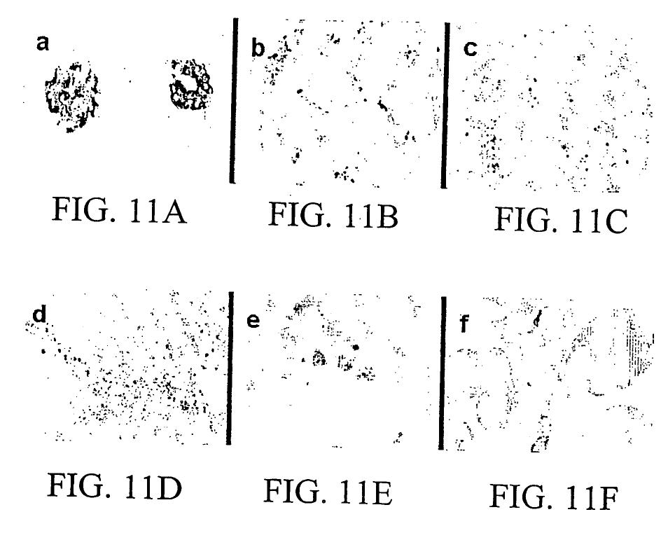

Application of immunohistochemistry for characterization of IPC generated from embryonic bodies derived from HESC is demonstrated in FIGS. 11A-F. Immunohistochemistry was performed with anti-insulin antibodies using staining of normal human pancreas as a positive control (FIG. 11A; ×40) and EBs at 19 days with a non-immune control serum as a negative control (FIG. 11F; ×10). Following differentiation of pluripotent hESC under conditions of spontaneous differentiation, we could identify clusters of IPC, scattered throughout the EB that represent approximately 1-3% of the population of cells within the EBs (FIGS. 11B-D; ×40, 19 days after differentiation). Staining was localized at the cytoplasm of these cells (FIG. 11E; ×100, 19 days after differentiation).

RT-PCR analysis was performed for identification of the expression pattern of genes specific to β-cells. This analysis verified appearance of beta-cell markers associated with secretion of insulin into the medium.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, aid steps for carrying out various disclosed functions may take a variety of alternative forms without departing from the scope of the invention.

1. An enriched population of adult stem cells derived from stem cells comprising a plurality of committed progenitor cells stably transfected with a polynucleotide construct comprising a telomerase promoter element operably linked to a sequence encoding a selectable marker, wherein the progenitor cells express telomerase promoter activity.

2. The population of adult stem cells according to claim 1, wherein the polynucleotide construct further encodes a reporter gene.

3. The population of adult stem cells according to claim 2, wherein the reporter gene encodes an optically detectable gene product.

4. The population of adult stem cells according to claim 1, wherein the stem cells are of human origin.

5. The population of adult stem cells according to claim 4, wherein the stem cells are derived from human embryonic stem cells.

6. The population of adult stem cells according to claim 1, wherein the telomerase promoter is of human origin.

7. The population according to claim 1, wherein the selectable marker is an antibiotic resistance gene.

8. The population of adult stem cells according to claim 1 further transfected with a lineage specific promoter operably linked to a gene encoding a selectable marker.

9. The population of adult stem cells according to claim 8, wherein the selectable marker is an antibiotic resistance gene.

10. The population of adult stem cells according to claim 1, wherein the cells are committed to a cell lineage selected from the group consisting of cardiomyocytes, beta islet cells, neuronal cells, hepatic cells, chondrocytes, dermal and epidermal cells, connective tissue cells, dendritic cells, hematopoietic cells and any other differentiated cell type which is potentially useful in human cell replacement therapy.

11. The population of adult stem cells according to claim 8, wherein the cells are committed to a cell lineage selected from the group consisting of cardiomyocytes, beta islet cells, neuronal cells, hepatic cells, chondrocytes, dermal and epidermal cells, connective tissue cells, dendritic cells, hematopoietic cells and any other differentiated cell type which is potentially useful in human cell replacement therapy.

12. An isolated subpopulation of adult stem cells derived from stem cells comprising a plurality of committed progenitor cells stably transfected with a polynucleotide construct comprising a telomerase promoter element operably linked to a sequence encoding a selectable marker, wherein the progenitor cells express telomerase promoter activity.

13. The subpopulation of adult stem cells according to claim 12, wherein the polynucleotide construct further encodes a reporter gene.

14. The subpopulation of adult stem cells according to claim 13, wherein the reporter gene encodes an optically detectable gene product.

15. The subpopulation of adult stem cells according to claim 12 wherein the stem cells are of human origin.

16. The subpopulation of adult stem cells according to claim 15, wherein the stem cells are derived from human embryonic stem cells.

17. The subpopulation of adult stem cells according to claim 12, wherein the telomerase promoter is of human origin.

18. The subpopulation of adult stem cells according to claim 12, wherein the selectable marker is an antibiotic resistance gene.

19. The subpopulation of adult stem cells according to claim 12, further transfected with a lineage specific promoter operably linked to a gene encoding a selectable marker.

20. The subpopulation of adult stem cells according to claim 19, wherein the selectable marker is an antibiotic resistance gene.

21. The cell subpopulation of adult stem cells according to claim 12, wherein the committed progenitors are specific to a cell lineage selected from the group consisting of cardiomyocytes, beta islet cells, neuronal cells, hepatic cells, chondrocytes, dermal and epidermal cells, connective tissue cells, dendritic cells, hematopoietic cells and any other differentiated cell type which is potentially useful in human cell replacement therapy.

22. The cell subpopulation of adult stem cells according to claim 19, wherein the committed progenitors are specific to a cell lineage selected from the group consisting of cardiomyocytes, beta islet cells, neuronal cells, hepatic cells, chondrocytes, dermal and epidermal cells, connective tissue cells, dendritic cells, hematopoietic cells and any other differentiated cell type which is potentially useful in human cell replacement therapy.

23. A cloned population of adult stem cells derived from stem cells comprising a plurality of committed progenitor cells stably transfected with a polynucleotide construct comprising a telomerase promoter element operably linked to a sequence encoding a selectable marker, wherein the progenitor cells express telomerase promoter activity.

24. The population of adult stem cells according to claim 23, wherein the polynucleotide construct further encodes a reporter gene.

25. The population of adult stem cells according to claim 24, wherein the reporter gene encodes an optically detectable gene product.

26. The population of adult stem cells according to claim 23, wherein the stem cells are of human origin.

27. The population of adult stem cells according to claim 25, wherein the adult stem cells are derived from human embryonic stem cells.

28. The population of adult stem cells according to claim 23, wherein the telomerase promoter is of human origin.

29. The population of adult stem cells according to claim 23, wherein the selectable marker is an antibiotic resistance gene.

30. The population of adult stem cells according to claim 23, wherein the cells are further transfected with a lineage specific promoter operably linked to a gene encoding a selectable marker.

31. The population of adult stem cells according to claim 30, wherein the selectable marker is an antibiotic resistance gene.

32. The population of adult stem cells according to claim 23, wherein the committed progenitors are specific to a cell lineage selected from the group consisting of cardiomyocytes, beta islet cells, neuronal cells, hepatic cells, chondrocytes, dermal and epidermal cells, connective tissue cells, dendritic cells, hematopoietic cells and any other differentiated cell type which is potentially useful in human cell replacement therapy.

33. The population of adult stem cells according to claim 30, wherein the committed progenitors are specific to a cell lineage selected from the group consisting of cardiomyocytes, beta islet cells, neuronal cells, hepatic cells, chondrocytes, dermal and epidermal cells, connective tissue cells, dendritic cells, hematopoietic cells and any other differentiated cell type which is potentially useful in human cell replacement therapy.

34. A population of stem cells stably transfected with a polynucleotide construct comprising ectopic telomerase encoding sequences, the cells over-expressing ectopic telomerase, wherein said cells can differentiate while maintaining higher telomerase activity than the telomerase activity of differentiated cells derived from non-transfected stem cells.

35. The population of cells according to claim 34, wherein the stem cells are of human origin.

36. The population of cells according to claim 35, wherein the stem cells are derived from human embryonic stem cells.

37. The population of cells according to claim 34, wherein the telomerase is of human origin.

38. In cell replacement therapy, the improvement which comprises utilizing the enriched population of adult stem cells according to claim 1 for cell replacement.

39. In cell replacement therapy, the improvement which comprises utilizing the isolated subpopulation of cells according to claim 12 for cell replacement.

40. In cell replacement therapy, the improvement which comprises utilizing the cloned population of cells according to claim 23 for cell replacement.
